(19) United States
(12) Patent Application Publication
(10) Pub. No.: US 2019/0080262 A1
(43) Pub. Date: Mar. 14, 2019

(54) WORK DETERMINATION SYSTEM, LEARNING DEVICE, AND LEARNING METHOD

(71) Applicant: HITACHI, LTD., Tokyo (JP)

(72) Inventors: Toru YAZAKI, Tokyo (JP); Yutaka UEMATSU, Tokyo (JP); Toshiaki TAKAI, Tokyo (JP); Tsuyoshi TAMAKI, Tokyo (JP)

(21) Appl. No.: 16/029,859

(22) Filed: Jul. 9, 2018

(30) Foreign Application Priority Data
Sep. 13, 2017 (JP) 2017-175415

(51) Int. Cl. G06N 99/00; G09B 23/00; A61B 5/0488

(52) U.S. Cl. CPC G06N 99/005; A61B 5/0488; G09B 23/00

(57) ABSTRACT

A work determination system includes: a biological signal obtaining unit obtaining a biological signal of a worker from a sensor attached to the worker during work; a feature extraction computation unit computing a feature extraction of the obtained biological signal of the worker; a work determination unit determining a work of the worker on the basis of a comparison result between the computed feature extraction of the biological signal of the worker and learning data generated in advance; and a learning unit generating the learning data, wherein the learning unit generates a musculoskeletal model corresponding to each worker, generates a quasi biological signal by reproducing a work of a determination target with the musculoskeletal model, computes a feature extraction of the quasi biological signal, and generates the learning data by associating, with each worker, the work of the determination target and a feature extraction distribution of the quasi biological signal.

### CROSS-REFERENCE TO RELATED APPLICATION

The present application claims priority from Japanese application JP 2017-175415, filed on Sep. 13, 2017, the content of which is hereby incorporated by reference into this application.

### BACKGROUND OF THE INVENTION

### 1. Field of the Invention

The present invention relates to a work determination system, a learning device, and a learning method.

### 2. Description of the Related Art

Conventionally, at production sites of various products, various techniques have been proposed that can monitor which work in the production process is carried out by workers for the purpose of improving production quality, optimizing production plan (resource allocation), improving the safety of workers, and the like.

JP 2017-73076 A discloses a behavior determination device 1 “including: a preset model unit 41; a user model unit 51; an information obtaining unit 21; a feature extraction unit 34 for extracting feature information from predetermined information obtained by the information obtaining unit 21; a model generation unit 134 determining availability of feature information based on similarity degree information including similarity degree between the feature information and each preset model information stored in the preset model unit 41, the model generation unit 134 generating, when the feature information is available, user model information from the feature information; and a behavior recognition unit 35 recognizing behavior based on the feature information and at least the user model information.”

### SUMMARY OF THE INVENTION

If user model information (learning data) is generated for each user (worker) as in the invention described in the above-mentioned JP 2017-73076 A, accurate determination of the work of each worker can be made. However, to generate learning data, it takes a certain learning time for each work. For example, even for a simple work such as screw tightening, the learning takes several tens of seconds, and in a complex work, it takes several minutes for that learning. In addition, since the actual production process consists of hundreds of works, it takes a huge amount of time to generate labeled learning data for each worker.

The present invention has been made in view of such circumstances, and it is an object of the present invention to be able to generate, in a shorter time, learning data with which a work of a worker can be determined with high accuracy.

The present application includes a plurality of means for solving at least a part of the above-mentioned problems, which are, for example, as follows. To achieve the above object, a work determination system according to an aspect of the present invention includes: a biological signal obtaining unit configured to obtain a biological signal of a worker from a sensor attached to the worker during work; a feature extraction computation unit configured to compute a feature extraction of the obtained biological signal of the worker; a work determination unit configured to determine a work of the worker on the basis of a comparison result between the computed feature extraction of the biological signal of the worker and learning data generated in advance; and a learning unit configured to generate the learning data, in which the learning unit generates a musculoskeletal model corresponding to each worker, generates a quasi biological signal by reproducing a work of a determination target with the musculoskeletal model, computes a feature extraction of the quasi biological signal, and generates the learning data by associating, with each of the workers, the work of the determination target and a feature extraction distribution of the quasi biological signal.

According to the present invention, learning data with which a work of a worker can be determined with high accuracy can be generated in a short time. The problems, configurations, and effects other than those described above will be understood by the description of the embodiments below.

### BRIEF DESCRIPTION OF THE DRAWINGS

FIG. 1 is a block diagram showing a configuration example of a work determination system according to a first embodiment of the present invention;

FIGS. 2A to 2C are diagrams showing an example of a myoelectric sensor;

FIG. 3 is a diagram showing an example of a sensor table;

FIG. 4 is a diagram showing a specific example of a correspondence relationship between an electrode of the myoelectric sensor and a muscle bundle;

FIGS. 5A and 5B are diagrams for explaining generation of quasi myoelectric signal based on muscle activity data;

FIG. 6 is a flowchart illustrating an example of processing of a work determination system;

FIG. 7 is a flowchart for explaining an example of learning data generation processing;

FIG. 8 is a flowchart explaining an example of work determination processing;

FIG. 9 is a block diagram showing a configuration example of a work determination system according to a second embodiment of the present invention;

FIG. 10 is a block diagram showing a configuration example of a work determination system according to a third embodiment of the present invention;

FIG. 11 is a block diagram showing a configuration example of a work determination system according to a fourth embodiment of the present invention; and

FIG. 12 is a block diagram showing a configuration example of a computer.

### DESCRIPTION OF THE PREFERRED EMBODIMENTS

Hereinafter, a plurality of embodiments according to the present invention will be described with reference to the drawings. In all the drawings for explaining the embodiments, the same members are denoted by the same reference numerals in principle, and the repetitive description thereof will be omitted. In the following embodiments, it is to be understood that the constituent elements (including the element steps and the like) are not necessarily indispensable except in the case where it is explicitly stated and the case where it is considered to be obviously indispensable in principle. In addition, it is to be understood that, when expressions such as “made up with A”, “consisting of A”, “having A” and “including A” do not exclude elements other than the element “A” except for the case where it is specifically indicated that nothing other than the element “A” is included. Similarly, in the following embodiments, when referring to the shapes, positional relationships, and the like of a constituent element, etc., the shapes and the like which are substantially similar thereto are also to be included, except for the case where it is explicitly stated or the case where such is obviously except for the case where it is explicitly stated and the case where it is considered obviously to be unlikely in principle.

<Configuration Example of Work Determination System which is First Embodiment According to the Present Invention>

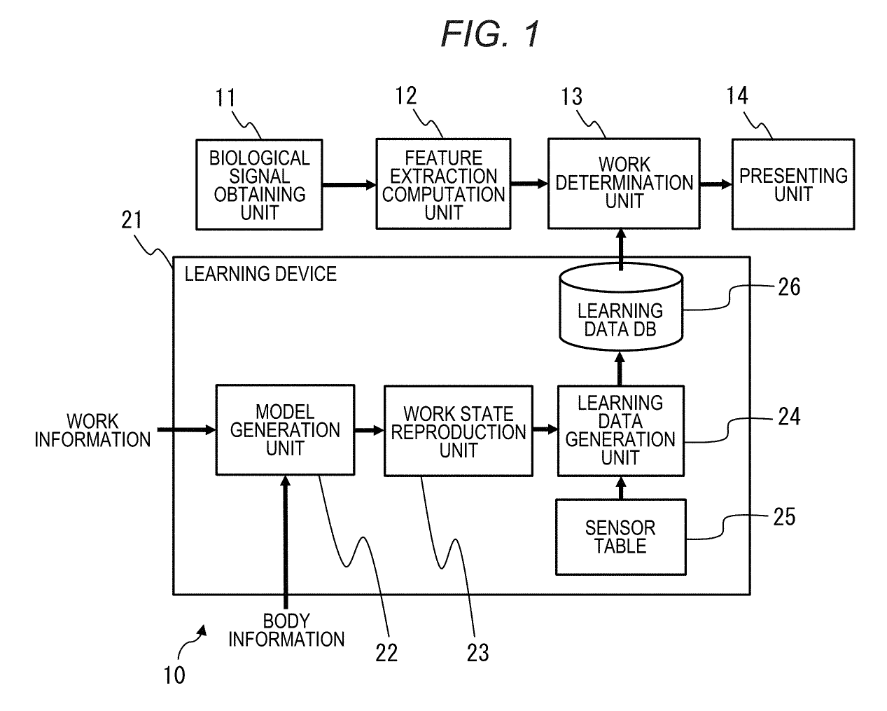

FIG. 1 is a block diagram showing a configuration example of a work determination system which is the first embodiment according to the present invention.

A work determination system 10 according to the first embodiment includes a biological signal obtaining unit 11, a feature extraction computation unit 12, a work determination unit 13, a presenting unit 14, and a learning device 21.

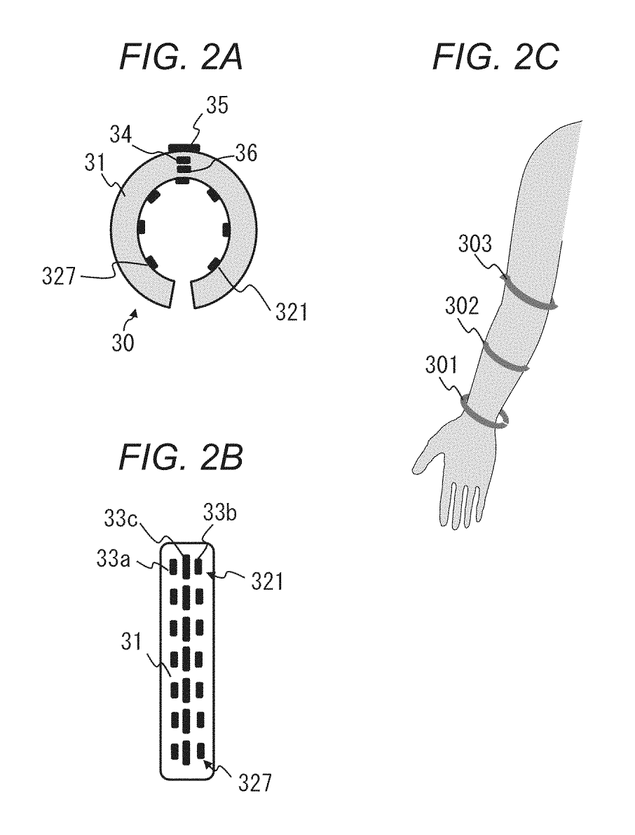

The biological signal obtaining unit 11 communicates with one or more my electromagnetic sensors (corresponding to a sensor of the present invention) 30 (FIGS. 2A to 2C) attached to the worker in accordance with a predetermined wireless communication specification, and receives myoelectric signal (corresponding to the biological signal of the present invention) the myoelectric sensor 30 received from the worker and supplies the electric signal to the feature extraction computation unit 12. Here, the myoelectric signal refers to a weak electric signal flowing through the nerve when muscle is moved.

The feature extraction computation unit 12 computes the feature extraction of the myoelectric signal by a predetermined feature extraction computation method for each section between the punctuation and punctuation of the myoelectric signal supplied from the biological signal obtaining unit 11 and notifies the feature extraction of the myoelectric signal to the work determination unit 13. Here, the punctuation of the myoelectric signal means, for example, a state in which the worker's work is stopped, so that the level of the myoelectric signal falls below a predetermined threshold or does not change. Note that the feature extraction of myoelectric signal may be computed every predetermined time (several seconds to several tens of seconds).

For any given feature extraction computation method, any existing method can be applied. The feature extraction computation method may include, for example, envelope detection, principal component analysis, and the like.

The work determination unit 13 compares the feature extraction of the myoelectric signal notified from the feature extraction computation unit 12 with the learning data previously generated and stored in the learning device (learning data DB 26). The work determination unit 13 determines the work that the worker is executing based on the comparison result with the learning data. More specifically, the work determination unit 13 compares the feature extraction of the myoelectric signal and all of the one or more feature extraction distributions associated with the worker to whom the myoelectric sensor 30 is attached, out of the learning data. Then, for example, the work corresponding to the feature extraction distribution closest to the feature extraction of the myoelectric signal is determined as the work that the worker is executing. Further, the work determination unit 13 notifies the presenting unit 14 of the determination result.

The presenting unit 14 presents the determination result notified from the work determination unit 13 (information representing work which the worker is executing), by, for example, outputting the determination result to a terminal used by an observer or the like monitoring the work of worker, or displaying the determination result on a display provided in the work determination system 10 (none is shown).

The operation from the biological signal obtaining unit 11 to the presenting unit 14 can be performed in real time at the same time as the work of the worker. However, it is also possible to record the myoelectric signal obtained by the myoelectric sensor 30 from the worker, determine and present the work executed by the worker based on the recorded myoelectric signal after the worker's work is completed.

The learning device 21 generates learning data to be compared with the feature extraction of the myoelectric signal in advance with the work determination unit 13 and trains the machine learning. Here, the learning data refers to one in which, for each worker, work of determination target and feature extraction distribution of quasi myoelectric signal are associated with each other (described in detail later).

The learning device 21 includes a model generation unit 22, a work state reproduction unit 23, a learning data generation unit 24, a sensor table 25, and a learning data DB (database) 26.

The model generation unit 22 generates a musculoskeletal model capable of reproducing a work with the work state reproduction unit 23 to be described later in accordance with work information supplied from the outside to the work determination system 10.

The model generation unit 22 corrects the musculoskeletal model in accordance with each worker based on the body information of each worker supplied from the outside to the work determination system 10. For example, if the height of the worker represented by the body information is higher than the generated musculoskeletal model, a correction is made to extend the musculoskeletal model according to the ratio of the height. Further, the model generation unit 22 supplies work information and the musculoskeletal model corrected corresponding to each worker to the work state reproduction unit 23.

Here, the work information is obtained by, for example, a state in which an arbitrary person is performing work of determination target by motion capture, and the work information is constituted by time-series three-dimensional coordinates of a plurality of markers attached to the body of the arbitrary person. Any person who performs the work of determination target may be anyone, for example, any person who performs the work of determination target may be selected from among a plurality of existing workers. Further, the work information is not limited to motion capture, and the work information may be obtained without using a marker by ranging, image processing, or the like. Further, the work information may be referred to as attitude information, motion information, or the like.

By providing a plurality of pieces of work information corresponding to the same work to the learning device 21, it is possible to further advance the training of the learning data and to improve the accuracy of the determination using the learning data.

The body information shall include at least one of body height, body weight, sex, muscle quantity, fat percentage, and skeleton information representing the body-like feature of each worker.

The musculoskeletal model is, for example, virtual model data of a bone, a joint, a skeletal muscle constituting a human body, and can be processed on a computer. For example, muscle activity data representing the movement of each skeletal muscle, motion, acceleration, angular velocity, etc. of a part such as a bone and a joint can be obtained by causing the musculoskeletal model to simulate a predetermined motion. Furthermore, human body load data representing loads on bones, joints, and skeletal muscle can be obtained.

With the musculoskeletal model corresponding to each worker supplied from the model generation unit 22, the work state reproduction unit 23 reproduces a work corresponding to work information, i.e., the work state reproduction unit 23 generates the muscle activity data (corresponding to musculoskeletal model output data according to the present invention) representing the movement of the skeletal muscle in the musculoskeletal model by causing the musculoskeletal model to simulate the work and supplies the muscle activity data to the learning data generation unit 24.

As described above, the muscle activity data represents the movement of the skeletal muscle constituting the musculoskeletal model. On the other hand, the myoelectric signal detected by the myoelectric sensor 30 is a weak electric signal generated in the nerve to move the skeletal muscle, so the muscle activity data and the myoelectric signal are different but are highly correlated with each other.

Therefore, in this embodiment, the result of performing predetermined computation on muscle activity data is deemed to be a quasi-like myoelectric signal. Hereinafter, the quasi-like myoelectric signal based on muscle activity data is referred to as quasi myoelectric signal (corresponding to quasi biological signal according to the present invention).

With the musculoskeletal model corresponding to each worker supplied from the model generation unit 22, the work state reproduction unit 23 simulates a work corresponding to work information, thereby generating human body load data representing loads on bones, joints, and skeletal muscle in the musculoskeletal model.

By referring to the sensor table 25, the learning data generation unit 24 generates a quasi myoelectric signal based on the muscle activity data supplied from the work state reproduction unit 23 (details will be described later). In addition, the learning data generation unit 24 computes the feature extraction of the generated quasi myoelectric signal using the same feature extraction computation method as the feature extraction computation unit 12 described above. Further, the learning data generation unit 24 generates learning data associating the work of determination target and the feature extraction distribution of quasi myoelectric signal with each worker by machine learning, deep learning, or the like, and stores the learning data in the learning data DB 26.

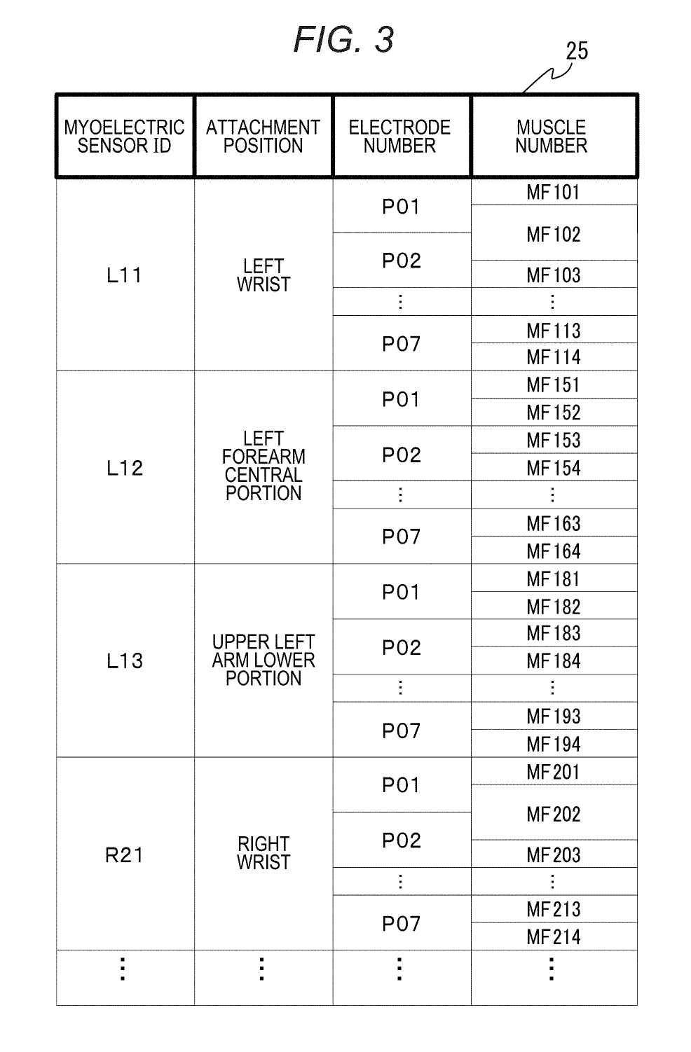

The sensor table 25 describes, for each myoelectric sensor 30 to be attached to the worker, the attachment position, the plurality of electrodes provided in the myoelectric sensor 30, and association with the skeleton muscle with which each electrode can detect the myoelectric signal (described in detail with reference to FIG. 3).

The learning data DB 26 holds the learning data generated by the learning data generation unit 24 and makes the work determination unit 13 refer to the held learning data.

<Configuration Example of Myoelectric Sensor 30>

Next, FIG. 2 shows the myoelectric sensor 30 attached to each worker. FIG. 2A is an example of an external view of the myoelectric sensor 30. FIG. 2B is an example of a developed view of the main body unit 31 of the myoelectric sensor 30. FIG. 2C shows an attachment example of the myoelectric sensor 30.

As shown in FIG. 2A, the myoelectric sensor 30 includes a main body unit 31 formed in an annular shape, a plurality of electrodes 32 (in the case of the figure, electrodes 321 to 327) arranged inside the main body unit 31 (the side where the worker touches) with a regular interval, a communication unit 34 arranged inside the main body unit 31, and a notification unit 35 arranged outside the main body unit 31.

The main body unit 31 is formed so as to be attached to the arm of the worker in close contact. As in the example shown in FIG. 2A, the annular shape of the main body unit 31 may be partially missing.

As shown in FIG. 2B, the electrode 32 is constituted by contact units 33a, 33b, 33c in contact with the skin of the worker. Out of the contact units 33a to 33c, the contact unit 33c is a reference potential, and the electrode 32 detects the potential difference between the contact units 33a and 33b as a myoelectric signal.

The communication unit 34 communicates with the biological signal obtaining unit 11 according to a predetermined wireless communication specification and transmits the myoelectric signal detected by each electrode 32.

The notification unit 35 notifies a worker, i.e., a user, of information indicating the operation status of the myoelectric sensor 30, the safety determination result of the work, and the like by using light, sound, letters, icons, and the like. Since the notification unit 35 is provided on the outside of the main body unit 31, the worker, i.e., the user, can move the myoelectric sensor 30 in the correct direction (for example, when attaching the myoelectric sensor 30 to the wrist, the notification unit 35 is attached so that it is on the back of the hand).

A motion detection unit 36 for detecting acceleration and angular velocity may be provided in the myoelectric sensor 30. In that case, the detected acceleration and angular velocity are transmitted to the biological signal obtaining unit 11 via the communication unit 34, and the feature extraction of the acceleration and angular velocity is computed by the feature extraction computation unit 12. On the other hand in the learning device 21, the work state reproduction unit 23 may compute the acceleration and angular velocity from the musculoskeletal model which reproduced the work, and the learning data generation unit 24 may compute the feature extraction of acceleration and angular velocity so that it is included in the learning data, and the feature extraction of acceleration and angular velocity may also be used for determination of the work.

For a worker, one or more myoelectric sensors 30 are attached. In the example shown in FIG. 2C, the myoelectric sensor 301 is attached to the worker's wrist, the myoelectric sensor 302 is attached to the central part of the forearm, and the myoelectric sensor 303 is attached to the lower part of the upper arm. The attachment position of the myoelectric sensor 30 is not limited to the example of FIG. 2C. For example, myoelectric sensor 30 may be attached to a leg of the worker.

Next, FIG. 3 shows an example of the sensor table 25. In the sensor table 25, the attachment position, the electrode number, and the muscle number representing the skeletal muscle in the human body and musculoskeletal model are recorded in association with the ID (identification information) of each myoelectric sensor 30 attached to the worker. It should be noted that the same muscle number may be associated with different electrodes 32. In that case, it means that the myoelectric signal from the skeletal muscle is detected by a plurality of different electrodes 32.

The example of FIG. 3 shows that, for example, the myoelectric sensor 30 with ID=L11 is attached to the left wrist and the electrode 32 (corresponding to the electrode 321) of the electrode number P01 out of the plurality of electrodes 32 provided in the myoelectric sensor 30 can detect the myoelectric signals from the muscle bundle of the muscle bundle numbers MF101, MF102. Similarly, it shows that the electrode 32 (corresponding to the electrode 322) of the electrode number P02 out of the plurality of electrodes 32 provided in the myoelectric sensor 30 can detect the myoelectric signals from the skeletal muscles of the muscle bundle numbers MF102, MF103.

Hereinafter, the myoelectric sensor 30 with ID=L11 is also referred to as a myoelectric sensor L11. The same applies to the myoelectric sensor 30 with ID=L12 and the like. The electrode 32 of the electrode number P01 is referred to as an electrode P01. The same applies to the electrodes 32 with the electrode numbers P02 to P07. Furthermore, the skeletal muscle of the skeletal muscle number MF101 is also referred to as the skeletal muscle MF101. The same applies to the skeletal muscle with the skeletal muscle number MF102 and the like.

Similarly, it is shown that the electrode P01 of the myoelectric sensor L12 can detect myoelectric signals from skeletal muscle MF151, MF152. Likewise, the following is shown. The electrode P02 of the myoelectric sensor L12 can detect myoelectric signals from skeletal muscles MF 153, MF 154.

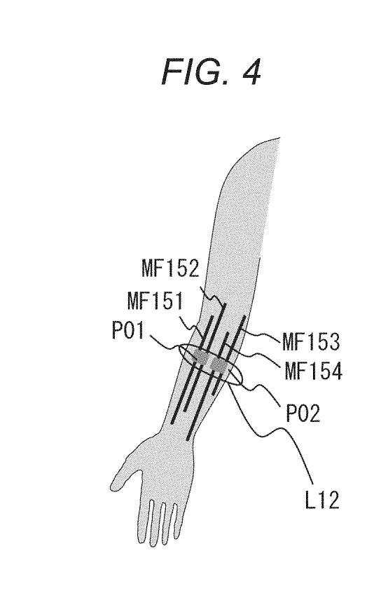

FIG. 4 is a diagram specifically showing the correspondence relationship between electrode P01 and skeletal muscles MF151, MF152, and the correspondence relationship between electrode P02 and skeletal muscle MF153, MF154.

That is, the electrode P01 of the myoelectric sensor L12 attached to the left forearm central portion of the worker can detect the myoelectric signals from the skeletal muscles MF151, MF152. In addition, the electrode P02 can detect the myoelectric signal from the skeletal muscles MF153, MF154.

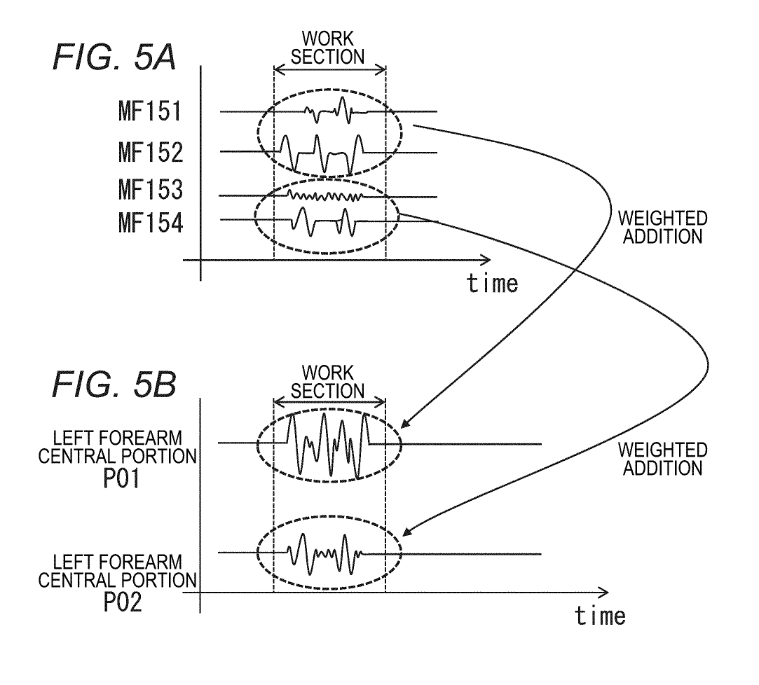

FIGS. 5A and 5B are diagrams for explaining processing for generating a quasi myoelectric signal based on the muscle activity data by the learning data generation unit 24 corresponding to the specific example of FIG. 4. FIG. 5A is muscle activity data obtained by reproducing work with the musculoskeletal model, and FIG. 5B shows quasi myoelectric signal generated based on muscle activity data.

That is, the learning data generation unit 24 generates a quasi myoelectric signal corresponding to the myoelectric signal actually detected by the electrode P01 of the myoelectric sensor L12 attached to the left forearm central portion of the worker in accordance with weighted addition in which each component corresponding to the skeletal muscles MF151, MF152 in the muscle activity data is multiplied by a predetermined weighting coefficient and then the products are added. In addition, the learning data generation unit 24 generates a quasi myoelectric signal corresponding to the myoelectric signal actually detected by the electrode P02 of the myoelectric sensor L12 in accordance with weighted addition in which each component corresponding to the skeletal muscles MF153, MF154 in the muscle activity data is multiplied by a predetermined weighting coefficient and then the products are added.

The weighting coefficients used to generate the quasi myoelectric signals based on the muscle activity data are predetermined, but the weighting coefficients may be changed by the user or may be corrected based on the measured value of the myoelectric signal (which will be explained as a second embodiment according to the present invention).

<Operation of Work Determination System 10 of the First Embodiment>

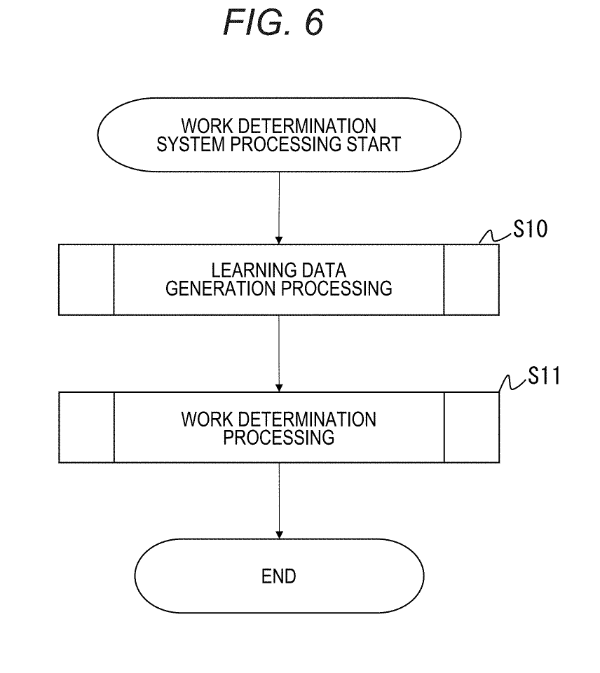

FIG. 6 is a flowchart for explaining the operation of the work determination system 10 which is the first embodiment.

That is, the operation of work determination system 10 is as follows: first, the learning device 21 executes learning data generation processing (step S10) for generating and holding learning data; and subsequently, the biological signal obtaining unit 11 to the presenting unit 14 execute the work determination processing (step S11). After learning data generation processing has been executed at least once, only work identification processing can be executed without going through learning data generation processing.

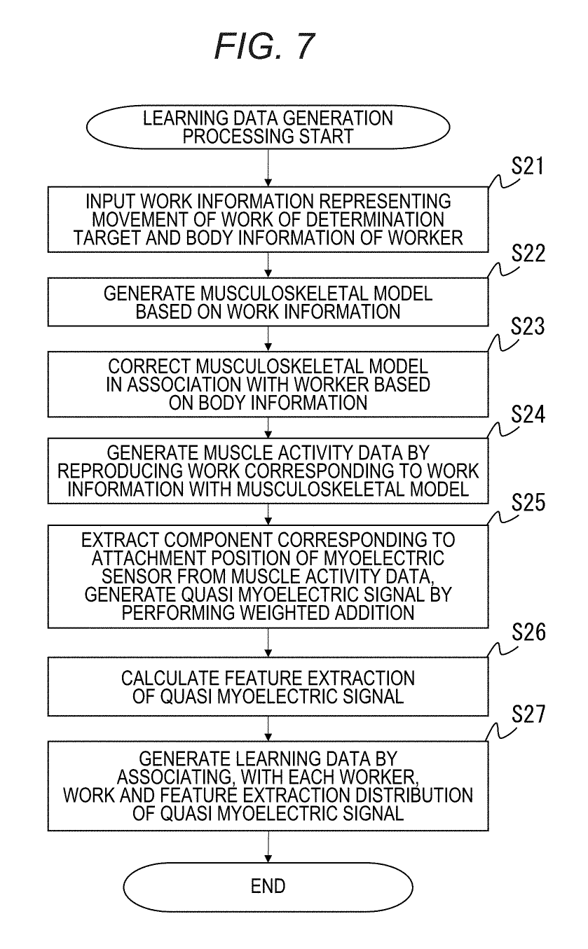

The learning data generation processing in step S10 will be described in detail. FIG. 7 is a flowchart illustrating learning data generation processing. The learning data generation processing is started, for example, in response to a start command from the user.

First, the model generation unit 22 accepts work information and body information supplied from the outside to the learning device 21 (step S21). Next, the model generation unit 22 generates a musculoskeletal model according to external work information (step S22). Next, the model generation unit 22 corrects the generated musculoskeletal model based on the body information of each worker, generates a musculoskeletal model corresponding to each worker, supplies the musculoskeletal model and work information corresponding to each worker thus generated to the work state reproduction unit 23 (step S23).

Next, with the musculoskeletal model corresponding to each worker supplied from the model generation unit 22, the work state reproduction unit 23 reproduces the work corresponding to the work information to generate muscle activity data, and supplies the muscle activity data to the learning data generation unit 24 (step S24).

Next, the learning data generation unit 24 refers to the sensor table 25 for each worker, thereby extracting the component corresponding to the attachment position of the myoelectric sensor 30 from the muscle activity data supplied from the work state reproduction unit 23 and applies weighted addition, thus generating the quasi myoelectric signal (step S25). Next, the learning data generation unit 24 computes the feature extraction of the generated quasi myoelectric signal (step S26). Next, the learning data generation unit 24 generates learning data in which the work of the determination target and the feature extraction distribution of the quasi myoelectric signal are associated with each worker, performs training, and holds it in the learning data DB 26 (step S27). Thus, the learning data generation processing is ended.

According to the learning data generation processing described above, even if each worker does not provide work information, it is possible to generate a learning model corresponding to each worker. Therefore, compared to the case where each worker provides work information, learning data can be generated in a shorter time. Also, the learning data can be generated for the work information without requiring the user or the like to perform labeling.

After the learning data is generated and held in the learning data DB 26 as described above, it is possible to execute the work determination processing of step S11.

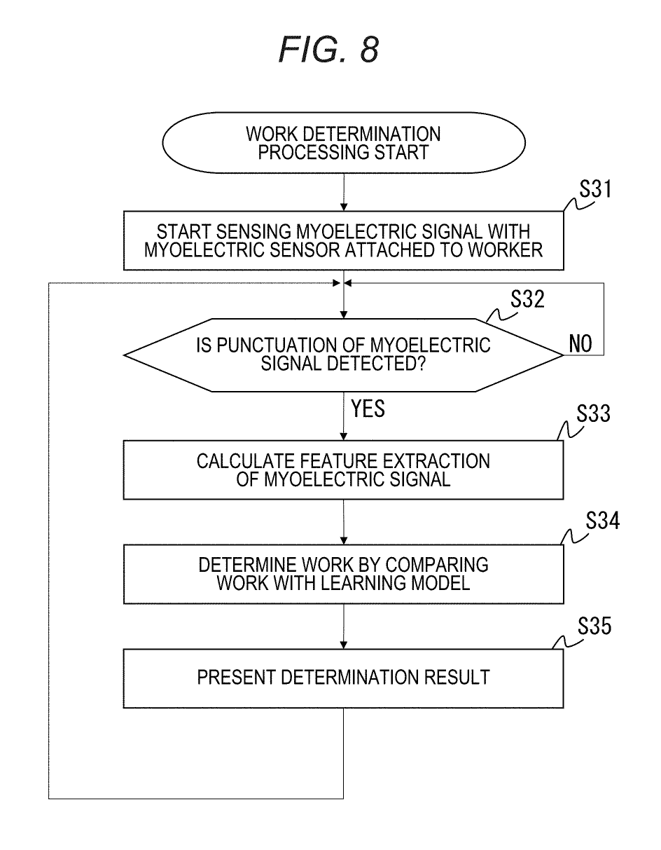

Next, FIG. 8 is a flowchart illustrating work determination processing. The work determination processing starts, for example, in response to a start command from a user.

First, the myoelectric sensor 30 attached to the worker starts sensing and transmitting the myoelectric signal. The biological signal obtaining unit 11 receives the myoelectric signals sequentially transmitted from the myoelectric sensor 30 and supplies the myoelectric signals to the feature extraction computation unit 12 (step S31).

The feature extraction computation unit 12 waits until it detects the punctuation of the myoelectric signal sequentially input from the biological signal obtaining unit 11 and accumulates the myoelectric signal (NO in step S32). Here, the punctuation of the myoelectric signal refers to the state in which the worker stops work, the state in which the level of the myoelectric signal is lower than or equal to the predetermined threshold, or the state in which the myoelectric signal does not change for a predetermined time. When the punctuation of the myoelectric signal is detected (YES in step S32), the feature extraction computation unit 12 computationally notifies the work determination unit 13 of the feature extraction of the myoelectric signal accumulated in the section up to the detected punctuation. Further, the feature extraction computation unit 12 clears the myoelectric signal for which the feature extraction has been computed (step S33).

Next, the work determination unit 13 compares the feature extraction of the myoelectric signal notified from the feature extraction computation unit 12 with the learning data held in the learning data DB 26, and determines the work that the worker executes based on the comparison result, and notifies the determination result to the presenting unit 14 (step S34).

Next, the presenting unit 14 presents a determination result (information representing a work that the worker is executing) notified from the work determination unit 13 by displaying the determination result on, for example, a display of a terminal (not shown) operated by an observer or the like who monitors the work of the worker (step S35).

Thereafter, while the worker having the myoelectric sensor 30 attached thereon is performing work, processing is returned to step S32, and steps S32 to S35 are repeatedly executed.

According to the work determination processing described above, the work which the worker is executing is determined by using the learning model corresponding to each worker, and therefore, it is possible to realize higher determination accuracy as compared with the case where the work is determined using a common learning model for each worker.

In the work determination processing described above, punctuation of myoelectric signal is detected and the work is determined by computing the feature extraction for each section between the detected punctuation and punctuation. Instead, the work may be determined based on a combination of feature quantities of myoelectric signals in each of a plurality of consecutive sections.

The feature extraction of the myoelectric signal may be computed to determine the work with every predetermined period of time, or the work may be determined based on a combination of feature quantities of the myoelectric signals every period of time of a predetermined consecutive period of times. After a work punctuation is roughly found from the moving image of the worker, the work may be determined based on the feature extraction of myoelectric signal.

<Configuration Example of Work Determination System which is Second Embodiment According to the Present Invention>

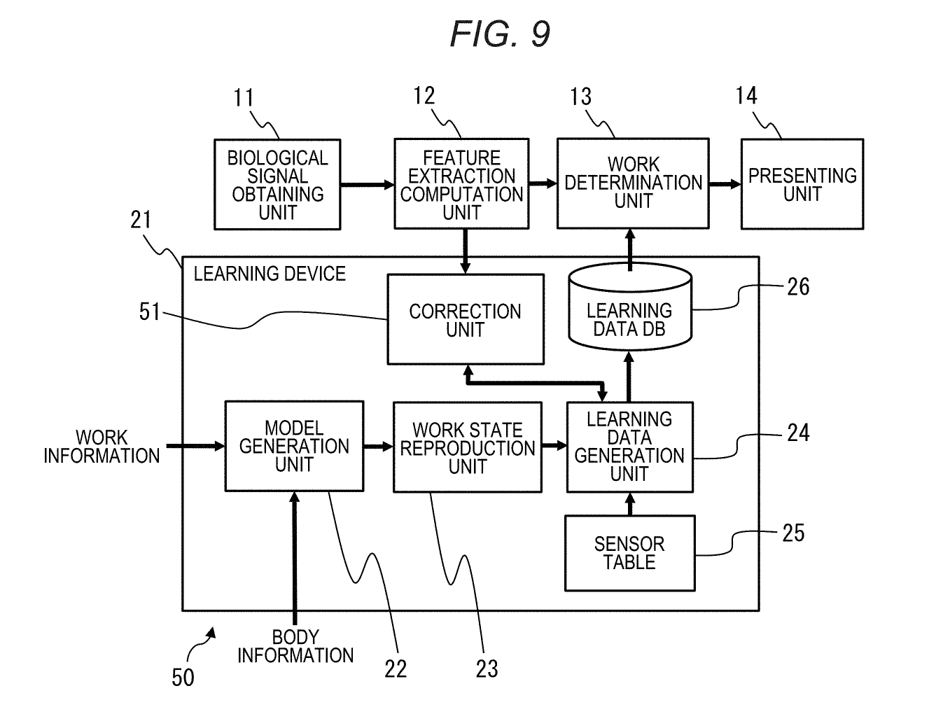

Next, FIG. 9 is a block diagram showing a configuration example of a work determination system which is the second embodiment according to the present invention.

A work determination system 50, which is the second embodiment, is obtained by adding a correction unit 51 to the learning device 21 of the work determination system 10 shown in FIG. 1. Out of the elements constituting the work determination system 50, the same reference numerals are assigned to elements common to the work determination system 10 which is the first embodiment, and the description thereof is omitted.

The correction unit 51 obtains from the feature extraction computation unit 12 the myoelectric signal when the worker performs a predetermined simple operation (for example, bending the elbow). Further, the correction unit 51 obtains from the learning data generation unit 24 the quasi myoelectric signal based on the muscle activity data obtained when reproducing the predetermined simple operation with the musculoskeletal model corresponding to the worker. Further, the correction unit 51 changes the weighting coefficients used for generating the quasi myoelectric signal so that the difference between the myoelectric signal and the quasi myoelectric signal is equal to or less than a predetermined threshold value. It is assumed that the quasi myoelectric signal is generated in advance for correction by the work state reproduction unit 23 and the learning data generation unit 24 and held by the learning data generation unit 24.

Furthermore, the correction unit 51 causes the learning data generation unit 24 to generate a quasi myoelectric signal using the changed weighting coefficient, re-compute the feature extraction, and correct and update the learning data for machine learning.

In the work determination system 50, after generating learning data by the learning data generation processing, the correction unit 51 executes the above-mentioned processing before performing the work determination processing.

According to the work determination system 50, by correcting the learning data, for example, it is possible to reduce the influence of the deviation of the attachment position of the myoelectric sensor 30 and the contact condition to the skin of the electrode 32 of the myoelectric sensor 30, etc. It is possible to improve the determination accuracy of work determination processing to be executed later.

<Configuration Example of Work Determination System which is Third Embodiment According to the Present Invention>

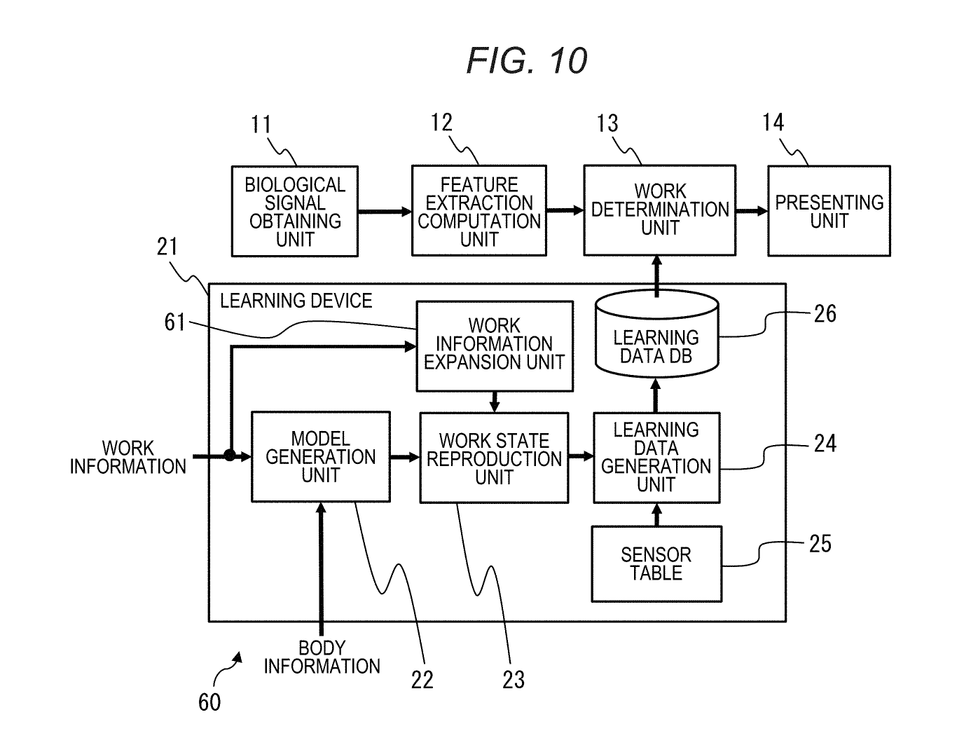

Next, FIG. 10 is a block diagram showing a configuration example of a work determination system which is a third embodiment according to the present invention.

The work determination system 60, which is the third embodiment, is obtained by adding a work information expansion unit 61 to the learning device 21 of the work determination system 10 shown in FIG. 1. Out of the elements constituting the work determination system 60, the same reference numerals are given to elements common to the work determination system 10 which is the first embodiment, and description thereof will be omitted.

In the work determination system 60, the work information supplied from the outside to the learning device 21 is also supplied to the work information expansion unit 61. The work information expansion unit 61 changes, by a predetermined variation range, at least one of the time of the supplied work information (for example, time-series three-dimensional coordinates of a plurality of markers attached to the body of an arbitrary person) and the three-dimensional coordinates, thereby generating a plurality of pieces of work information corresponding to the same work on the basis of external work information. More specifically, for example, even with the same work for tightening the screw, a plurality of pieces of work information are generated from a single piece of work information by changing the time required, slightly changing the position of the joint, the angle of the hand, and the like. Further, the work information expansion unit 61 supplies a plurality of pieces of work information corresponding to the same work generated to the work state reproduction unit 23.

In the work state reproduction unit 23, a work corresponding to each pieces of work information corresponding to the same work supplied from the work information expansion unit 61 can be reproduced with the musculoskeletal model to generate a plurality of pieces of muscle activity data, and the generated plurality of pieces of muscle activity data can be supplied to the learning data generation unit 24. As a result, the number of pieces of muscle activity data supplied to the learning data generation unit 24 increases.

Therefore, according to the work determination system 60, without supplying a plurality of pieces of work information corresponding to the same work from the outside, it is possible to increase the number of pieces of work information corresponding to the same work and supply them to the learning data generation unit 24, so that a larger number of learning data can be generated. Therefore, it is possible to improve the determination accuracy of the work determination processing using the obtained learning data.

<Configuration Example of Work Determination System which is Fourth Embodiment According to the Present Invention>

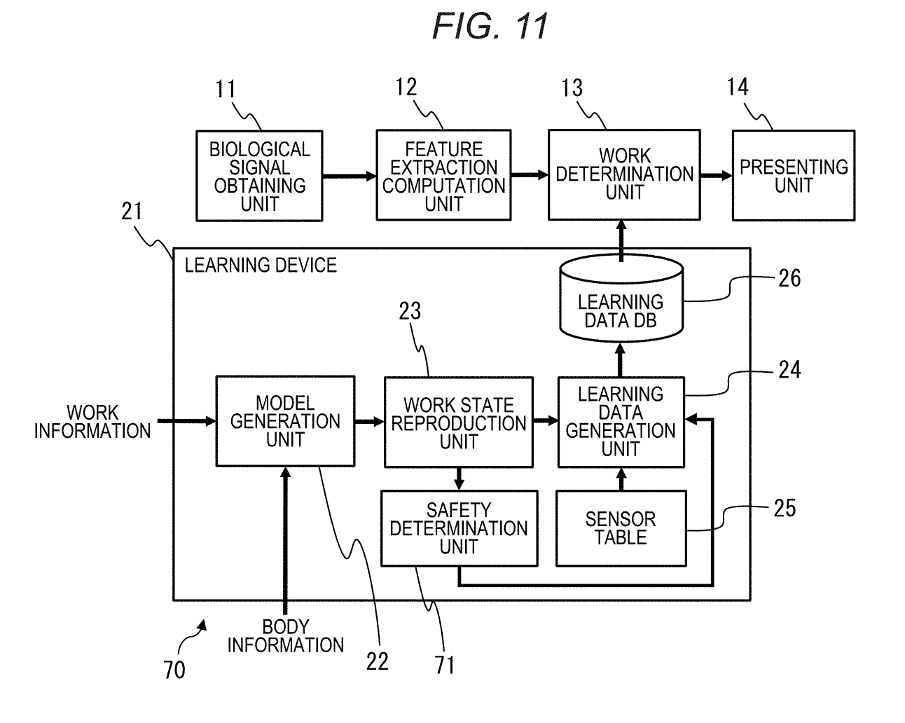

Next, FIG. 11 is a block diagram showing a configuration example of a work determination system which is a fourth embodiment according to the present invention.

The work determination system 70 which is the fourth embodiment is obtained by adding a safety determination unit 71 to the learning device 21 of the work determination system 10 shown in FIG. 1. Out of the elements constituting the work determination system 70, the same reference numerals are assigned to elements common to the work determination system 10 which is the first embodiment, and the description thereof is omitted.

In the work determination system 70, the work state reproduction unit 23 reproduces the work corresponding to work information with the musculoskeletal model corresponding to each worker, thereby generating the human body load data representing the load on each bone, each joint, each muscle bundle in the musculoskeletal model. Further, the work state reproduction unit 23 supplies the generated human body load data to the safety determination unit 71.

Based on the human body load data supplied from the work state reproduction unit 23, the safety determination unit 71 determines the safety when the worker executes the work corresponding to the work information and sends the determination result to the learning data generation unit 24.

Incidentally, the safety determination result of the safety determination unit 71 includes, for example, “do not continue this work for 1 hour or more, please take a break”, “this work has the risk of hurting your back” as well as precautions to be considered when executing the work.

The learning data generation unit 24 records the determination result of safety by the safety determination unit 71 in association with the generated learning data. Then, when the work performed by the worker is determined by the work determination processing, the work determination unit 13 notifies the determination result of safety as well as the determination result of work to the presenting unit 14. The presenting unit 14 presents the determination result of safety as well as the determination result of work by displaying them, for example on the display. Further, the work determination unit 13 may transmit the determination result of safety to the myoelectric sensor 30 attached to the worker via the biological signal obtaining unit 11. The notification unit 35 of the myoelectric sensor 30 may alert the worker by light, sound, characters, icons, or the like.

According to work determination system 70, not only the work that the worker is doing but also the safety of the work can be determined, and the determination result can be notified to the worker etc., so that only the work efficiency can be improved but also worker's safety can be ensured.

It should be noted that the first to fourth embodiments of the present invention described above can be appropriately combined.

By the way, the work determination system 10 or the like which is the embodiment of the present invention described above can be configured by hardware or can be realized by software. When the work determination system 10 or the like is realized by software, a program constituting the software is installed in the computer. Here, the computer includes a computer incorporated in dedicated hardware, and a general-purpose personal computer or the like capable of executing various functions by installing various programs, for example.

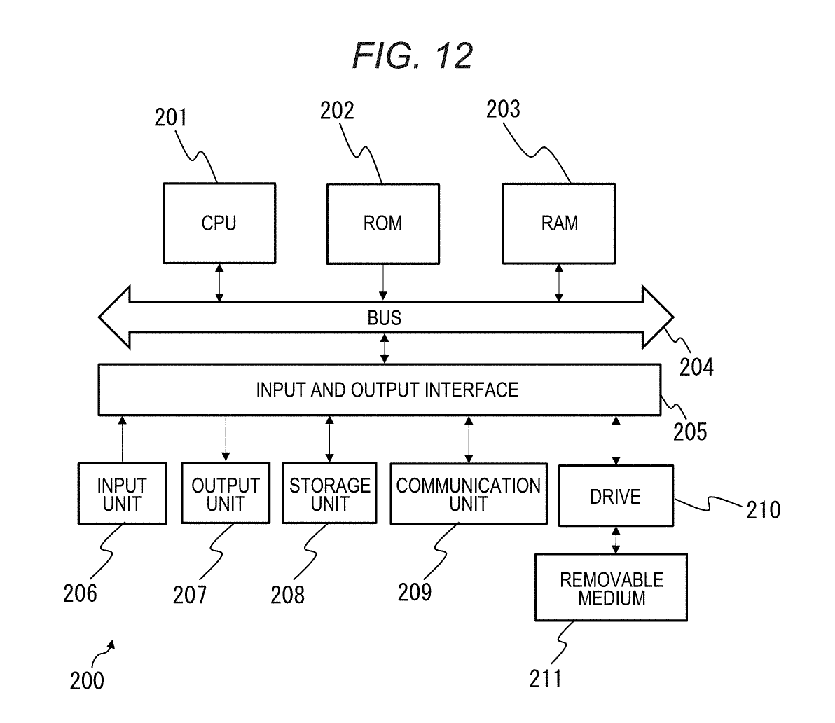

FIG. 12 is a block diagram showing a configuration example of hardware of a computer realizing a work determination system 10 and the like with a program.

In the computer 200, a central processing unit (CPU) 201, a read only memory (ROM) 202, and a random access memory (RAM) 203 are connected with each other by a bus 204.

An input and output interface 205 is further connected to the bus 204. An input unit 206, an output unit 207, a storage unit 208, a communication unit 209, and a drive 210 are connected to the input and output interface 205.

The input unit 206 is composed of a keyboard, a mouse, a microphone, and the like. The output unit 207 is composed of a display, a speaker, and the like. The storage unit 208 is composed of a hard disk, a nonvolatile memory, or the like. The communication unit 209 is composed of a network interface or the like. The drive 210 drives a removable medium 211 such as a magnetic disk, an optical disk, a magneto-optical disk, or a semiconductor memory.

In the computer 200 configured as described above, for example, the CPU 201 loads a program stored in the storage unit 208 into the RAM 203 via the input and output interface 205 and the bus 204 and executes the program, whereby the work determination system 10 etc. are realized.

More specifically, the biological signal obtaining unit 11 of the work determination system 10 can communicate with the myoelectric sensor 30 via the communication unit 209, for example. The presenting unit 14 can output a determination result to a terminal or display which presents the determination result of work via the output unit 207 or the communication unit 209. The learning device 21 can request work information and body information via the input unit 206, the communication unit 209, the drive 210, and the like.

The program executed by the computer 200 (CPU 201) can be provided by being recorded in the removable medium 211 as a package medium or the like, for example. In addition, the program can be provided via a wired or wireless transmission medium such as a local area network, the Internet, digital satellite broadcasting or the like.

In the computer 200, the program can be installed in the storage unit 208 via the input and output interface 205 by attaching the removable medium 211 to the drive 210. In addition, the program can be received by the communication unit 209 via a wired or wireless transmission medium and installed in the storage unit 208. In addition, the program can be installed in the ROM 202 or the storage unit 208 in advance.

It should be noted that the program executed by the computer 200 may be a program in which processing is performed in chronological order according to the order described in this specification, or may be a program in which processing is performed in parallel or at a necessary timing such as when a call is made.

The effects described in this specification are merely examples and are not limited, and other effects may be provided.

The present invention is not limited to the embodiments described above, but includes various modifications. For example, each of the above-described embodiments has been described in detail in order to explain the present invention in an easy-to-understand manner, and the present invention is not necessarily limited to one having all the constituent elements described. A part of the configuration of one embodiment can be replaced by the configuration of another embodiment, and the configuration of another embodiment can be added to the configuration of any embodiment. Further, it is possible to add, delete, and replace other configurations with respect to a part of the configuration of each embodiment.

The above-described configurations, functions, processing units, processing means, and the like may be realized in hardware by designing some or all of configurations, functions, processing units, processing means, and the like, for example, by an integrated circuit. Each of the above-described configurations, functions, and the like may be realized by software by interpreting and executing a program that realizes the respective functions by the processor. Information such as a program, table, file, and the like that realizes each function can be stored in a memory device, a storage device such as a hard disk, a solid state drive (SSD), or a recording medium such as an IC card, an SD card, or a DVD. In addition, control lines and information lines indicate what is considered to be necessary for explanation, and not necessarily all control lines and information lines are necessarily shown on the product. In practice, it can be considered that almost all the structures are mutually connected.

The present invention can be provided not only in the work determination system, the learning device, and learning method but also in various aspects such as a computer readable program executed with a learning device, a work determination method by a work determination system, and the like.

What is claimed is:

1. A work determination system comprising:
a biological signal obtaining unit configured to obtain a biological signal of a worker from a sensor attached to the worker during work;
a feature extraction computation unit configured to compute a feature extraction of the obtained biological signal of the worker;
a work determination unit configured to determine a work of the worker on the basis of a comparison result between the computed feature extraction of the biological signal of the worker and learning data generated in advance; and
a learning unit configured to generate the learning data,
wherein the learning unit generates a musculoskeletal model corresponding to each worker, generates a quasi biological signal by reproducing a work of a determination target with the musculoskeletal model, computes a feature extraction of the quasi biological signal, and generates the learning data by associating, with each of the workers, the work of the determination target and a feature extraction distribution of the quasi biological signal.

2. The work determination system according to claim 1, wherein the learning unit includes:
a model generation unit generating the musculoskeletal model on the basis of work information representing the work of the determination target and body information about each worker;
a work state reproduction unit generating musculoskeletal model output data by reproducing the work corresponding to the work information with the generated musculoskeletal model; and
a learning data generation unit generating a quasi biological signal using the generated musculoskeletal model output data, computes a feature extraction of the quasi biological signal, and generates the learning data by associating, with each of the workers, the work of the determination target and the feature extraction distribution of the quasi biological signal.

3. The work determination system according to claim 2, wherein the learning data generation unit generates the quasi biological signal by performing predetermined computation by extracting a component corresponding to an attachment position of the sensor of the musculoskeletal model output data generated.

4. The work determination system according to claim 2, wherein the learning data generation unit extracts a component corresponding to an attachment position of the sensor from the generated musculoskeletal model output data and applies weighted addition, thus generating the quasi biological signal.

5. The work determination system according to claim 2, wherein
the biological signal is a myoelectric signal, and
the musculoskeletal model output data is muscle activity data.

6. The work determination system according to claim 2, wherein the body information includes at least one of height, weight, sex, muscle quantity, fat percentage, and skeleton information.

7. The work determination system according to claim 2, wherein the model generation unit generates a musculoskeletal model as the musculoskeletal model based on the work information obtained through motion capture performed on any person executing the work of the determination target and the body information of each worker.

8. The work determination system according to claim 2, wherein the learning unit includes a correction unit corrects the learning data generated in advance on the basis of a comparison result between the biological signal of the worker detected by the sensor attached to the worker during the work and the quasi biological signal which has been generated in advance.

9. The work determination system according to claim 2, wherein
the learning unit includes a work information expansion unit providing a predetermined variation range in the input work information, and
the work state reproduction unit generates musculoskeletal model output data by reproducing a work corresponding to the work information, for which the variation range is provided, with the generated musculoskeletal model.

10. The work determination system according to claim 1, comprising a presenting unit presenting a determination result given by the work determination unit.

11. The work determination system according to claim 2, wherein
the work state reproduction unit also generates human body load data by reproducing a work corresponding to the work information with the generated musculoskeletal model, and
the learning unit includes a safety determination unit determining safety of a work corresponding to the work information on the basis of the human body load data.

12. The work determination system according to claim 11, comprising a presenting unit presenting a determination result provided by the work determination unit and presenting a determination result presented by the safety determination unit.

13. The work determination system according to claim 1, wherein the sensor includes:
a main body unit; and
a plurality of electrodes arranged, with a regular distance, on the main body unit.

14. The work determination system according to claim 1, wherein
the sensor detects at least one of acceleration, and angular velocity,
the feature extraction computation unit computes at least one feature extraction of the acceleration and the angular velocity detected, and
the work determination unit determines a work of the worker on the basis of not only the feature extraction of the biological signal of the worker computed but also at least one feature extraction of the acceleration and the angular velocity computed and the comparison result with learning data generated in advance.

15. A learning device comprising
a model generation unit generating a musculoskeletal model corresponding to each worker;
a work state reproduction unit generating musculoskeletal model output data by reproducing the work of a determination target with the generated musculoskeletal model; and
a learning data generation unit generating a quasi biological signal using the generated musculoskeletal model output data, computes a feature extraction of the quasi biological signal, and generates learning data by associating, with each of the workers, the work of the determination target and the feature extraction distribution of the quasi biological signal.

16. A learning method of a learning device, wherein the learning device executes:
generating a musculoskeletal model corresponding to each worker;
generating musculoskeletal model output data by reproducing the work of a determination target with the generated musculoskeletal model; and
generating a quasi biological signal using the generated musculoskeletal model output data, computes a feature extraction of the quasi biological signal, and generates learning data by associating, with each of the workers, the work of the determination target and the feature extraction distribution of the quasi biological signal.
